(19) United States
(12) Patent Application Publication
(10) Pub. No.: US 2021/0178045 A1
(43) Pub. Date: Jun. 17, 2021

(54) FLOW CHAMBER WITH HELICAL FLOW PATH

(71) Applicant: Fresenius Medical Care Holdings, Inc., Waltham, MA (US)

(72) Inventors: Irving Hernandez, Waltham, MA (US); Diego Suarez del Real, Waltham, MA (US); Pedro Almeida, Waltham, MA (US)

(21) Appl. No.: 16/717,221

(22) Filed: Dec. 17, 2019

(51) Int. Cl. A61M 1/36

(52) U.S. Cl. CPC A61M 1/3627; A61M 1/1627; A61M 2206/20; A61M 2206/16

(57) ABSTRACT

A dialysis system, such as a hemodialysis system, includes a flow chamber. The flow chamber includes: a tube section having a first end and a second end, a tube section longitudinal axis extending between the first end and the second end, the tube section having an inner wall and outer wall; and a helical flow path disposed in the inner wall of the tube section, the helical flow path extending along at least a portion of the tube section longitudinal axis.

### FIELD

Exemplary embodiments of the invention relate to a flow chamber for use in, for example, a hemodialysis system. The flow chamber has a helical flow path.

### BACKGROUND

Patients with kidney failure or partial kidney failure typically undergo hemodialysis treatment in order to remove toxins and excess fluids from their blood. In hemodialysis treatment, blood is taken from the dialysis patient through an intake needle or catheter which draws blood from an artery or vein located in a specifically accepted access location, for example, a shunt surgically placed in an arm, thigh, subclavian artery, or the like. The needle or catheter is connected to extracorporeal tubing that is fed to a peristaltic pump and then to a dialyzer that cleans the blood and removes excess fluid. The dialyzed blood is then returned to the patient through additional extracorporeal tubing and another needle or catheter. Sometimes, a heparin drip is located in the hemodialysis loop to prevent the blood from coagulating.

As the drawn blood passes through the dialyzer, it travels in straw-like tubes within the dialyzer that serve as semi-permeable passageways for the unclean blood. Fresh dialysate solution enters the dialyzer at its downstream end. The dialysate surrounds the straw-like tubes and flows through the dialyzer in the opposite direction of the blood flowing through the tubes. Fresh dialysate collects toxins passing through the straw-like tubes by diffusion and excess fluids in the blood by ultra filtration. Dialysate containing the removed toxins and excess fluids is disposed of as waste. The red cells remain in the straw-like tubes and their volume count is unaffected by the process.

It is desirable to avoid mixing air into the blood when the blood is outside of the patient's body, as the presence of air in the blood can have various negative consequences for the patient when the dialyzed blood is returned to the patient's body. Accordingly, hemodialysis systems may also include one or more components intended to separate entrained air from the blood.

### SUMMARY

A flow chamber for use in a dialysis treatment is provided. The flow chamber can include a tube section having a first end and a second end. A tube section longitudinal axis extends between the first end and the second end. The tube section has an inner wall and outer wall. A helical flow path disposed in the inner wall of the tube section. The helical flow path extends along at least a portion of the tube section longitudinal axis.

In an embodiment of the flow chamber, the helical flow path extends radially outward from the inner wall of the tube section.

In an embodiment of the flow chamber, the helical flow path has a rounded cross-section. In an embodiment of the flow chamber, the helical flow path has a hemispherical cross-section.

In an embodiment of the flow chamber, the tube section has a first outer diameter at the first end of the tube section and a second outer diameter at the second end of the tube section, the first outer diameter being greater than the second outer diameter.

In an embodiment of the flow chamber, the tube section tapers from the first end of the tube section to the second end of the tube section.

In an embodiment of the flow chamber, the helical flow path extends from the first end of the tube section to the second end of the tube section.

In an embodiment of the flow chamber, the flow chamber further includes a flow inlet disposed at the first end of the tube section. In an embodiment of the flow chamber, the helical flow path extends into the flow inlet.

In an embodiment of the flow chamber, the flow chamber further includes a flow outlet disposed at the second end of the tube section.

In an embodiment of the flow chamber, the helical flow path is at a first angle with respect to the tube section longitudinal axis. In an embodiment of the flow chamber, the first angle is 75°.

In an embodiment of the flow chamber, the helical flow path includes a first helical flow path portion at a first angle with respect to the tube section longitudinal axis and a second helical flow path portion adjacent to the first helical flow path portion. The second helical flow path portion is at a second angle with respect to the tube section longitudinal axis. The second angle is different than the first angle. In an embodiment of the flow chamber, the second angle is greater than the first angle.

A fluid management system for use in a dialysis treatment is also provided. The fluid management system can include a flow chamber. The flow chamber can include a tube section having a first end and a second end. A tube section longitudinal axis extends between the first end and the second end. The tube section has an inner wall and outer wall. A flow inlet is disposed at the first end of the tube section. A flow outlet is disposed at the second end of the tube section. A helical flow path is disposed in the inner wall of the tube section. The helical flow path extends along at least a portion of the tube section longitudinal axis. The fluid management system can also include an end cap arranged on the flow inlet.

In an embodiment of the fluid management system, the helical flow path extends radially outward from the inner wall of the tube section.

In an embodiment of the fluid management system, the helical flow path has a rounded cross-section.

In an embodiment of the fluid management system, the tube section has a first outer diameter at the first end of the tube section and a second outer diameter at the second end of the tube section, the first outer diameter being greater than the second outer diameter.

In an embodiment of the fluid management system, the helical flow path extends from the first end of the tube section to the second end of the tube section.

### BRIEF DESCRIPTION OF THE DRAWINGS

Exemplary embodiments of the present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 is a schematic diagram of a hemodialysis system including a flow chamber according to an exemplary embodiment of the invention;

FIG. 2 shows a perspective view of a flow chamber according to an exemplary embodiment of the invention;

FIG. 3 shows a cross-sectional view of the flow chamber of FIG. 2 along line 3-3;

FIG. 4 shows a perspective view of an embodiment of a flow chamber with a flow outlet according to an exemplary embodiment of the invention;

FIG. 5 shows a cross-sectional view of the flow chamber of FIG. 4 along line 5-5;

FIG. 6 shows an end view of a flow chamber according to an exemplary embodiment of the invention;

FIG. 7 shows a perspective view of a flow chamber according to an exemplary embodiment of the invention;

FIG. 8 shows a perspective view of a fluid management system according to an exemplary embodiment of the invention;

FIG. 9 shows a cross-sectional view of the fluid management system of FIG. 8 along line 9, 10-9, 10, wherein the helical flow path of the tube section does not extend into the flow inlet;

FIG. 10 shows a cross-sectional view of the fluid management system of FIG. 8 along line 9, 10-9, 10, wherein the helical flow path of the tube section extends into the flow inlet;

FIG. 11 shows a cross-sectional view of the fluid management system of FIG. 9 with fluid therein;

FIG. 12 shows a flow chamber according to another exemplary embodiment of the invention, wherein a helical flow path of the flow chamber has helical flow path portions at two different angles with respect to the tube section longitudinal axis; and

FIG. 13 shows a cross-sectional view of the flow chamber of FIG. 4 along line 5-5 with exemplary dimensions.

### DETAILED DESCRIPTION

Exemplary embodiments of the present invention provide a flow chamber with improved fluid management. The flow chamber may be used, for example, in a hemodialysis system, which dialyzes blood. The flow chamber reduces oxygenation of the dialyzed blood before the dialyzed blood is returned to the dialysis patient. The flow chamber also minimizes coagulation of the blood therein, and, correspondingly, the risk of introducing a blood clot in the patient upon return of the dialyzed blood to the patient.

The flow chamber of exemplary embodiments of the present invention provides improved fluid management through the provision of a helical flow path in an inner wall of a tube section of the flow chamber. In practice, the flow chamber receives, in drop form, dialyzed blood at a first end of the flow chamber. For example, at the beginning of a dialysis session with a patient, the dialyzed blood begins to accumulate within the flow chamber so as to partially fill the flow chamber with dialyzed blood. Eventually the flow of blood into and out of the flow chamber reaches an approximately steady state, such that the flow chamber is partially filled with blood and the remainder of the flow chamber is filled with air.

The helical flow path is disposed in an inner wall of the tube section of the flow chamber. In an embodiment, the helical flow path can be formed by debossing the inner wall of the tube section. In this manner, the helical flow path extends radially outward from the center of the tube section such that the inner diameter of the tube section at the helical flow path is increased due to the presence of the helical flow path. The helical flow path can have a rounded cross-section. The helical flow path can also have hemispherical cross-section. A rounded cross-section may be desirable because it reduces the creation of additional turbulence within the flow in the flow chamber. However, in other exemplary embodiments, the cross-section of the helical flow path may not be rounded.

As the blood drips into the flow chamber, the drops fall onto the helical flow path in the inner wall of the tube section, either directly contacting at least one of the inner wall or the helical flow path or after a minimal free fall distance within the flow chamber. The drops then progress, at least in part, along the helical flow path. In this manner, the helical flow path reduces the velocity of the drops as they progress through the tube section. Reducing the velocity of the drops helps to minimize the formation of foam that would occur within the flow chamber if the drops were allowed to free fall for longer distances or if the drops moved at a faster velocity. It is desirable to limit the formation of foam within the blood chamber so as to minimize coagulation of the blood and blood clots within the flow chamber.

The flow chamber according to exemplary embodiments of the present invention may be fitted with a flow inlet at the first end of the flow chamber. The flow inlet can act as an extension of the flow chamber. In an embodiment, the helical flow path can extend into the flow inlet, lengthening the helical flow path. The flow inlet may be similar in structure to the flow chamber in that the flow inlet is also tubular. The flow inlet may also be tapered in the same manner as the flow chamber. The flow inlet can be made of the same or a different material as the flow chamber.

An end cap can be attached either to the flow chamber or to the flow inlet if the flow chamber is provided with a flow inlet. The end cap includes one or more ports that facilitate fluidic connection of the flow chamber to the hemodialysis system vis-à-vis extracorporeal tubing.

The flow chamber may be fitted with a flow outlet at the second end of the flow chamber. The flow outlet allows the flow chamber to be connected to standard diameter extracorporeal tubing. Dialyzed blood flows from the flow chamber, through the flow outlet, into the extracorporeal tubing, and then into the return needle or catheter so that the dialyzed blood can be returned to the patient. The flow outlet may be similar in structure to the flow chamber in that the flow outlet is also tubular, at least in part. Accordingly, the flow outlet may also be tapered in the same manner as the flow chamber. The flow outlet then transitions to a nozzle shape to facilitate connection to the standard diameter extracorporeal tubing. The flow outlet can be made of the same or a different material as the flow chamber.

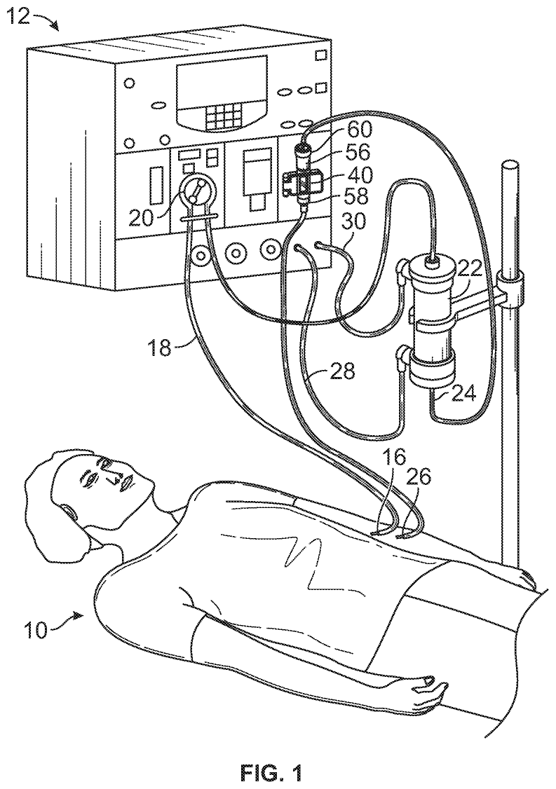

FIG. 1 is a schematic diagram of a hemodialysis system in which a patient 10 is undergoing hemodialysis treatment using a hemodialysis machine 12. An input needle or catheter 16 is inserted into an access site of the patient 10, such as in the arm, and is connected to extracorporeal tubing 18 that leads to a peristaltic pump 20 and to a dialyzer 22 (or blood filter). The dialyzer 22 removes toxins and excess fluid from the patient's blood. The excess fluids and toxins are removed by clean dialysate liquid which is supplied to the dialyzer 22 via a tube 28, and waste liquid is removed for disposal via a tube 30. The dialyzed blood is returned to the patient 10 from the dialyzer 22 through the extracorporeal tubing 24 and a return needle or catheter 26. In the context of exemplary embodiments of the present invention, a flow chamber 40 is fluidically disposed between the extracorporeal tubing 24 and the return needle or catheter 26. The flow chamber 40 can include a flow inlet 56, a flow outlet 58, and an end cap 60, as discussed in further detail below, so as to provide a fluid management system.

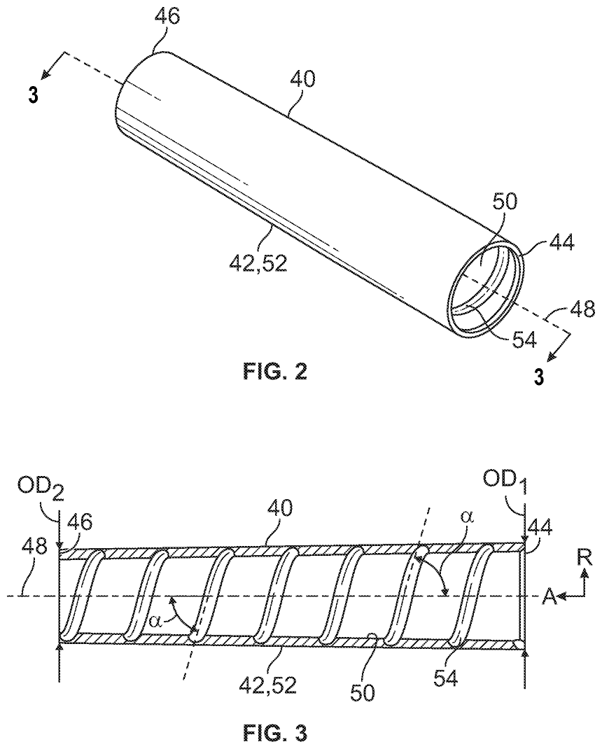

FIG. 2 shows the flow chamber 40. The flow chamber 40 comprises a tube section 42, which has a first end 44 and a second end 46. The second end 46 is disposed opposite the first end 44 on tube section 42. A tube section longitudinal axis 48 extends along tube section 42 between the first end 44 and the second end 46. The tube section 42 has an inner wall 50 and outer wall 52. A thickness of the tube section (i.e., a distance between the inner wall 50 and the outer wall 52) is relatively small compared to an overall diameter of the tube section 42. For example, in an embodiment, a thickness of the tube section 42 (e.g., at first end 44) could be 1651 μm±127 μm.

FIG. 3 shows the flow chamber 40 of FIG. 2 in cross-section along line 3-3 in FIG. 2. The tube section longitudinal axis 48 defines an axial direction A, to which radial direction R is perpendicular. A helical flow path 54 is disposed in the inner wall 50 of the tube section 42. The helical flow path 54 extends along at least a portion of the tube section longitudinal axis 48. In this embodiment, the helical flow path 54 extends over an entire length of tube section 42 (i.e., from first end 44 to second end 46). The helical flow path 54 extends radially outward from the inner wall 50 of tube section 42 (i.e., in a radial direction R with respect to tube section longitudinal axis 48). In this manner, the helical flow path 54 forms a recessed channel in the inner wall 50 of the tube section 42. For example, in an embodiment, the helical flow path extends radially outward 952.5 μm±12.7 μm from the inner wall 50 of the tube section 42. The helical flow path 54 helps reduce the velocity of drops of blood that enter the flow chamber 40, as discussed above.

As seen in FIG. 3, the tube section 42 has a first outer diameter OD₁ at its first end 44 and a second outer diameter OD₂ at its second end 46. The first outer diameter OD₁ is greater than the second outer diameter OD₂. The outer wall 52 and the inner wall 50 of the tube section 42 can taper, or continuously narrow, from its first end 44 to its second end 46. For example, in an embodiment, the angle or slope of the taper is 1°±0.2°. Such narrowing of the tube section 42 along its length (i.e., along axial direction A) also facilitates reducing the velocity of drops of blood that enter the flow chamber 40 by ensuring that blood drops input into the flow chamber 40 (e.g., from drip outlet 68 as shown in FIG. 9) will directly contact at least one of the inner wall 50 or the helical flow path 54 without free falling through the flow chamber 40 or after a minimal free fall distance within the flow chamber 40.

FIG. 3 shows the helical flow path 54 at a first angle α with respect to the tube section longitudinal axis 48. Adjusting the first angle α of the helical flow path 54 with respect the tube section longitudinal axis 48 affects how quickly the flow chamber 40 reduces the velocity of drops of blood as the drops progress through the tube section 42. In general, a helical flow path 54 having smaller first angle α will cause the flow chamber 40 to more gradually reduce the velocity of the drops than if the helical flow path 54 has a larger first angle α. Conversely, a helical flow path 54 having larger first angle α will cause the flow chamber 40 to more quickly reduce the velocity of the drops than if the helical flow path 54 has a smaller first angle α.

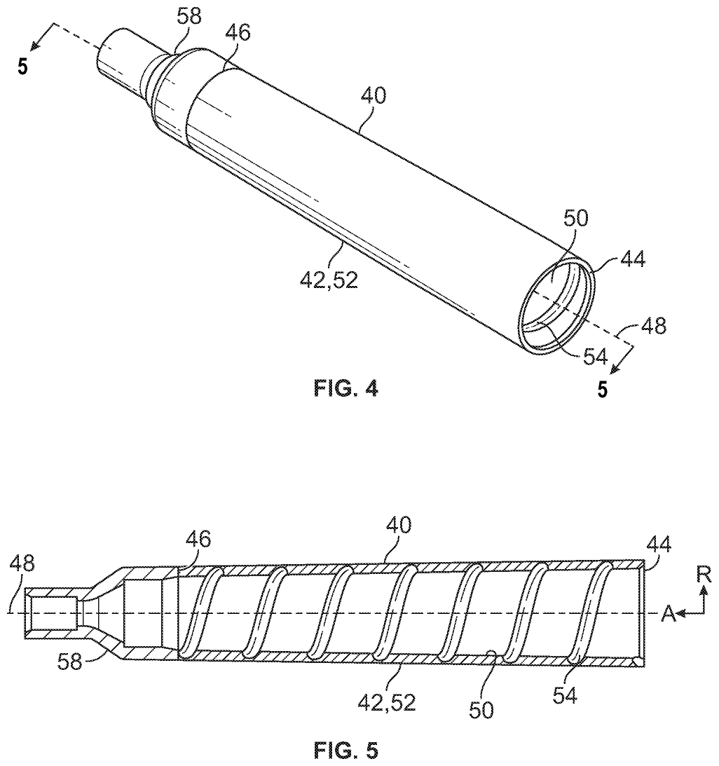

FIG. 4 shows the flow chamber 40 with a flow outlet 58 disposed at the second end 46 of the tube section 42. The flow outlet 58 further narrows the passageway through which the dialyzed blood flows. The end of the flow outlet 58 that does not abut the second end 46 of the tube section 42 may be attached to tubing so as to fluidically transport dialyzed blood from the flow chamber 40 to the return needle or catheter 26, as shown in FIG. 1. FIG. 5 shows the flow chamber 40 of FIG. 4 in cross-section along line 5-5 in FIG. 4.

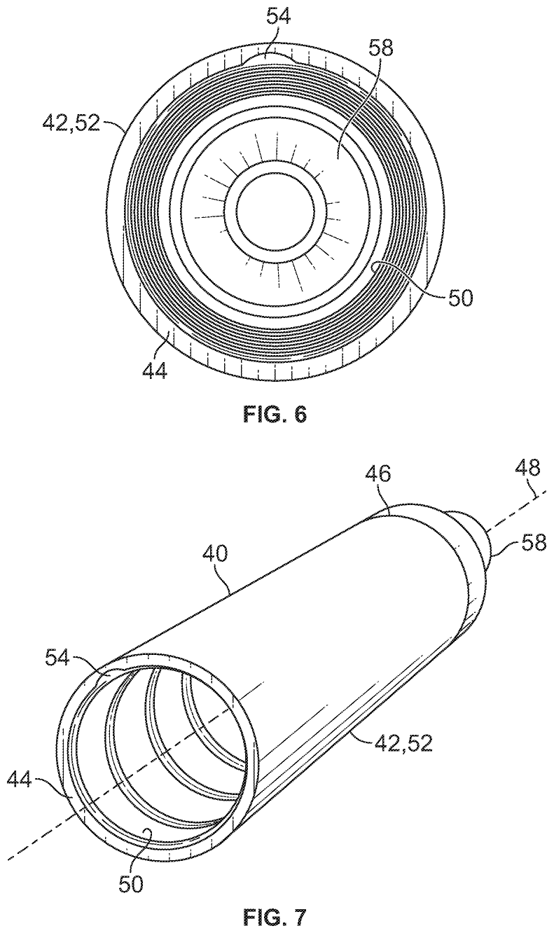

As shown in FIGS. 6-7, the helical flow path 54 has a rounded cross-section. A rounded cross-section helps mitigate turbulence within the flow of the blood within the tube section 42. The cross-sectional geometry of the helical flow path 54 can vary. For example, the helical flow path 54 can have a hemispherical cross-section.

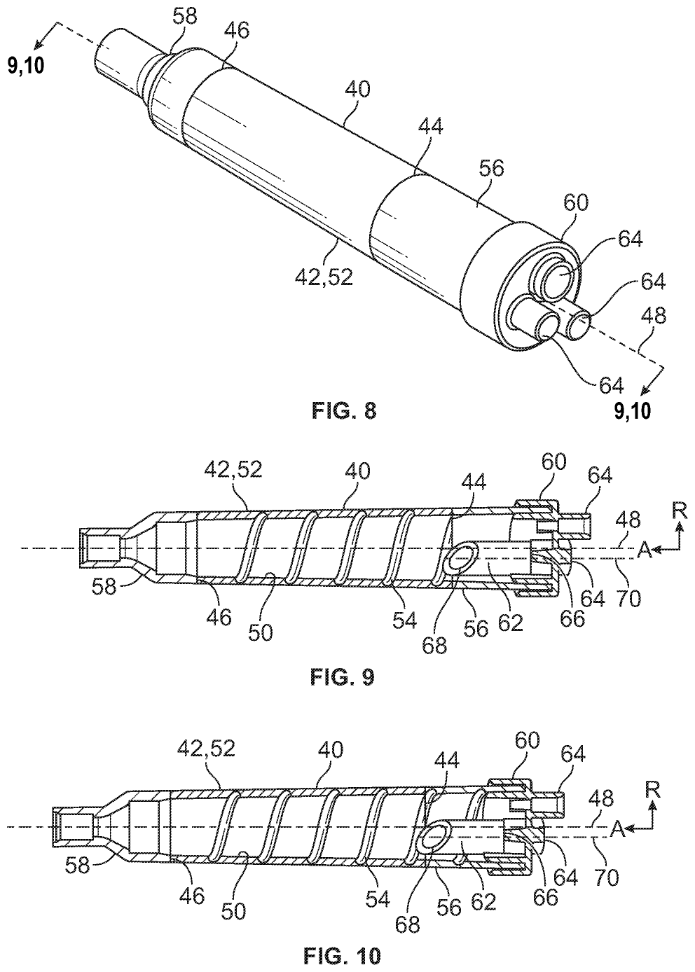

FIG. 8 shows a fluid management system according to an exemplary embodiment of the invention, the fluid management system comprising the flow chamber 40 with the flow outlet 58 disposed at the second end 46 of the tube section 42, a flow inlet 56 disposed at the first end 44 of the tube section 42, and an end cap 60 arranged on the flow inlet 56. The flow inlet 56 is a longitudinal extension of the tube section 42, in that the flow inlet 56 acts as a continuation of both the inner wall 50 and the outer wall 52 of the tube section 42. The end cap 60 facilitates connection of the flow chamber 40 to extracorporeal tubing 24, as shown in FIG. 1. The end cap 60 is secured to the flow inlet 56, for example, by tolerance fit. Alternatively, in the absence of the flow inlet 56, the end cap 60 may be secured to the first end 44 of the tube section 42, for example, by tolerance fit.

FIGS. 9-10 show the fluid management system of FIG. 8 in cross-section along line 9, 10-9, 10 in FIG. 8. As shown in FIGS. 9-10, the end cap 60 includes a drip tube 62 having a drip tube inlet 66 and a drip tube outlet 68. A drip tube longitudinal axis 70 extends between the drip tube inlet 66 and the drip tube outlet 68. The drip tube longitudinal axis 70 is parallel to the tube section longitudinal axis 48. The drip tube 62 is positioned radially outward (i.e., in radial direction R) from the tube section longitudinal axis 48. In this manner, the drip tube 62 is positioned such that the drip tube outlet 68 is proximal to or in contact with the inner wall 50 of the flow chamber 40. This helps to ensure that blood drops input into the flow chamber 40 from drip outlet 68 will directly contact at least one of the inner wall 50 or the helical flow path 54 without free falling through the flow chamber 40 or after a minimal free fall distance within the flow chamber 40. Then, immediately or soon after the drops enter the first end 44 of the tube section 42, the drops are carried along the helical flow path 54 so as to reduce the velocity of the drops as they progress through the tube section 42, as previously discussed.

As shown in FIG. 9, the helical flow path 54 of tube section 42 ends at the first end 44 of the tube section 42 such that the helical flow path 54 does not extend into the flow inlet 56. In FIG. 10, in contrast, the helical flow path 54 extends into the flow inlet 56, such that inclusion of the flow inlet 56 in the fluid management system can lengthen the helical flow path 54.

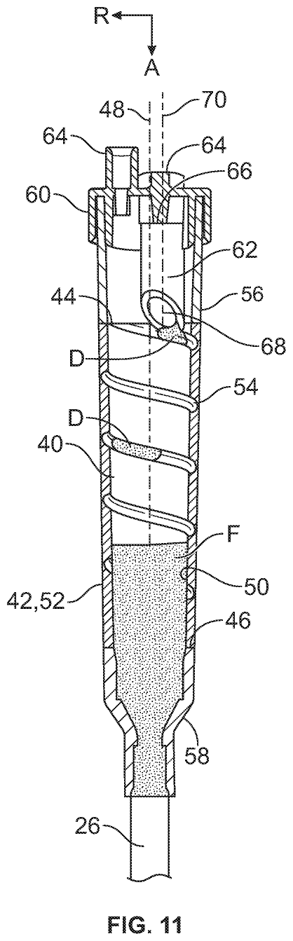

FIG. 11 shows the fluid management system of FIG. 9 in operation. Drops D exit the drip tube 62 at drip tube outlet 68, moving in the axial direction A (i.e., from the first end 44 of the tube section 42 toward the second end 46 of the tube section 42). The drops D directly contact at least one of the inner wall 50 of the tube section 42 or the helical flow path 54 without free falling through the flow chamber 40 or after a minimal free fall distance within the flow chamber 40, reducing the velocity of the drops D and minimizing the creation of foam within the flow chamber 40. After a number of the drops D enter the flow chamber 40, fluid F, which comprises drops D, accumulates in the lower portion of the flow chamber 40. The fluid F ultimately exits the flow chamber 40 through the flow outlet 58 and passes to the return needle or catheter 26 to be returned to the patient 10, as shown in FIG. 1.

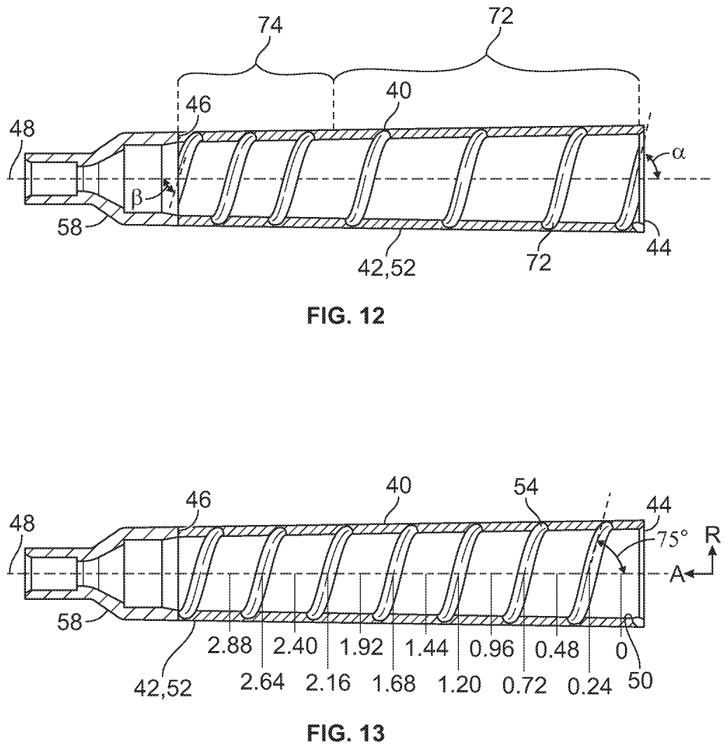

FIG. 12 shows another embodiment of the flow chamber according to an exemplary embodiment of the invention. In contrast to the embodiment shown in FIG. 3, the helical flow path 54 comprises a first helical flow path portion 72 arranged adjacent to a second helical flow path portion 74. The first helical flow path portion 72 is at a first angle α with respect to the tube section longitudinal axis 48 while the second helical flow path portion 74 is at a second angle β with respect to the tube section longitudinal axis 48. The second angle β is different than the first angle α. For example, in an embodiment, the second angle β is greater than the first angle α. Varying the first angle α and the second angle β affects how quickly the flow chamber 40 reduces the velocity of drops of blood as the drops progress through the tube section 42, as described in connection with FIG. 3. For example, in an embodiment, the first angle α is 75°±2° and the second angle β is 80°±2°.

In an alternative embodiment, the first angle α with respect to the tube section longitudinal axis 48 can, over the length of the tube section 42 (i.e., from first end 44 to second end 46), gradually change to the second angle β so that the helical flow path 54 provides a smooth reduction in velocity of the drops as the drops progress through the flow chamber 40.

FIG. 13 shows the flow chamber 40 of FIG. 4 in cross-section along line 5-5 in FIG. 4, adding exemplary dimensions in centimeters and degrees. The exemplary dimensions are intended to be illustrative and not limiting in any way. For example, in the embodiment of the flow chamber 40 shown in FIG. 13, a turn-to-turn distance along tube section longitudinal axis 48 from the center of one turn of the helical flow path 54 to the center of an adjacent turn of the helical flow path 54 along axial direction A is 0.48 cm, while the first angle α is 75°. The turn-to-turn distance can range from 0.4673 cm-0.4927 cm, while the first angle α can range from 73°-77°. If FIG. 13 were not shown in cross-section, a distance from the center of one turn of the helical flow path 54 to the center of an adjacent turn of the helical flow path 54 along axial direction A would be half as much, namely 0.24 cm, due to the presence of the helical flow path 54 on the other half of the flow chamber 40.

While exemplary embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. For example, the present invention includes further embodiments with any combination of features from the different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A flow chamber, comprising:
a tube section having a first end and a second end, a tube section longitudinal axis extending between the first end and the second end, the tube section having an inner wall and outer wall; and
a helical flow path disposed in the inner wall of the tube section, the helical flow path extending along at least a portion of the tube section longitudinal axis.

2. The flow chamber of claim 1, wherein the helical flow path extends radially outward from the inner wall of the tube section.

3. The flow chamber of claim 1, wherein the helical flow path has a rounded cross-section.

4. The flow chamber of claim 3, wherein the helical flow path has a hemispherical cross-section.

5. The flow chamber of claim 1, wherein the tube section has a first outer diameter at the first end of the tube section and a second outer diameter at the second end of the tube section, the first outer diameter being greater than the second outer diameter.

6. The flow chamber of claim 5, wherein the tube section tapers from the first end of the tube section to the second end of the tube section.

7. The flow chamber of claim 1, wherein the helical flow path extends from the first end of the tube section to the second end of the tube section.

8. The flow chamber of claim 1, further comprising a flow inlet disposed at the first end of the tube section.

9. The flow chamber of claim 8, wherein the helical flow path extends into the flow inlet.

10. The flow chamber of claim 1, further comprising a flow outlet disposed at the second end of the tube section.

11. The flow chamber of claim 1, wherein the helical flow path is at a first angle with respect to the tube section longitudinal axis.

12. The flow chamber of claim 11, wherein the first angle is 75°.

13. The flow chamber of claim 1, wherein the helical flow path comprises:
a first helical flow path portion at a first angle with respect to the tube section longitudinal axis; and
a second helical flow path portion adjacent to the first helical flow path portion, the second helical flow path portion being at a second angle with respect to the tube section longitudinal axis, the second angle being different than the first angle.

14. The flow chamber of claim 13, wherein the second angle is greater than the first angle.

15. A fluid management system, comprising:
a flow chamber, the flow chamber comprising:
a tube section having a first end and a second end, a tube section longitudinal axis extending between the first end and the second end, the tube section having an inner wall and outer wall;
a flow inlet disposed at the first end of the tube section;
a flow outlet disposed at the second end of the tube section; and
a helical flow path disposed in the inner wall of the tube section, the helical flow path extending along at least a portion of the tube section longitudinal axis, and an end cap arranged on the flow inlet.

16. The fluid management system of claim 15, wherein the end cap comprises a drip tube having a drip tube inlet and a drip tube outlet, a drip tube longitudinal axis extending between the drip tube inlet and the drip tube outlet, and
wherein the drip tube longitudinal axis is parallel to and disposed radially outward from the tube section longitudinal axis.

17. The fluid management system of claim 15, wherein the helical flow path extends radially outward from the inner wall of the tube section.

18. The fluid management system of claim 15, wherein the helical flow path has a rounded cross-section.

19. The fluid management system of claim 15, wherein the tube section has a first outer diameter at the first end of the tube section and a second outer diameter at the second end of the tube section, the first outer diameter being greater than the second outer diameter.

20. The fluid management system of claim 15, wherein the helical flow path extends from the first end of the tube section to the second end of the tube section.
